(19) United States
(12) Patent Application Publication
(10) Pub. No.: US 2023/0084684 A1
(43) Pub. Date: Mar. 16, 2023

(54) SYSTEM AND METHOD FOR ACCREDITATION OF INDUSTRIAL PROFESSIONALS

(71) Applicant: Rajeshwari Kartik, Allen, TX (US)

(72) Inventor: Rajeshwari Kartik, Allen, TX (US)

(21) Appl. No.: 17/476,629

(22) Filed: Sep. 16, 2021

(51) Int. Cl. G06Q 50/20; G06Q 10/10; G06Q 10/06; G16H 40/20; G06Q 30/00; G06F 16/951; G06F 16/9535

(52) U.S. Cl. CPC G06Q 50/2057; G06Q 10/105; G06Q 10/06398; G06Q 10/1093; G16H 40/20; G06Q 30/018; G06F 16/951; G06F 16/9535; G06Q 20/027

(57) ABSTRACT

A system for accreditation of industrial professionals is disclosed. The plurality of subsystems includes a profile generation subsystem, configured to create user profiles for one or more registered users using one or more user credentials. The plurality of subsystems includes an event manager subsystem, configured to provide access to continued industrial education content to the one or more registered users based on the created user profiles and also configured is manage one or more tasks of the user profiles relating to the content. The plurality of subsystems also includes a credit generation subsystem, configured to generate credit scores for the user profiles based on the one or more tasks completed by the one or more registered users. The plurality of subsystems also includes a report generation subsystem configurate to generate and publish a report on the one or more tasks.

### FIELD OF INVENTION

Embodiments of the present disclosure relates to the technical field of information management systems, and more particularly to a system and a method for accreditation of industrial professionals.

### BACKGROUND

Today every industrial field is developing at a rapid pace and the associated depth of knowledge is increasing every moment. Therefore, it becomes essential for an industrial professional to continuously become acquainted with the latest developments in their respective industrial field. The industrial field can include medical, engineering, legal, and the like. The rapid pace of industrial advances makes it mandatory for professionals to keep themselves updated such that they may apply this information to their practice and thus fortify their competence and knowledge by staying updated with latest developments in their field.

The industrial professionals need to maintain their knowledge, skills, competence and performance over time and hence, they need to continue their industrial education. Many jurisdictions require continuing industrial education for each industrial professional to maintain their industrial license. For example, continuing medical education (CME) is one such type of Continuing Industrial Education. Such education methods are based on credit points or hours. Various educational councils have accepted such methods of learning to provide licences and accreditation.

Conventionally, there is a lack of a common platform for industrial professionals and industrial organizations and institutes in accessing information related to continuing industrial education and accreditation related that the same. Moreover, systems currently available lack the integration of varied data sources for access by industrial professionals. Real time calculation mechanism for the credit points or hours are also desirable for distributing industrial licenses.

Hence, there is a need for an improved system for the accreditation of industrial professionals and a method to operate the same and therefore address the aforementioned issues.

### BRIEF DESCRIPTION

In accordance with one embodiment of the disclosure, a system for accreditation of industrial professionals is disclosed. The system comprising a hardware processor. The system a memory coupled to the hardware processor. The memory comprises a set of program instructions in the form of a plurality of subsystems. The plurality of subsystems is configured to be executed by the hardware processor.

The plurality of subsystems includes a profile generation subsystem. The profile generation subsystem is configured to create a user profile for each of one or more registered users using one or more user credentials. The plurality of subsystems includes an event manager subsystem. The event manager subsystem is configured is provide access to continuing industrial education content to the one or more registered users based on the created user profiles. The event manager subsystem is also configured is manage one or more tasks associated with the user profiles relating to the continuing industrial education content responsive to providing the access.

The plurality of subsystems also includes a credit generation subsystem. The credit generation subsystem is configured to generate a credit score for each of the user profiles based on the one or more tasks completed by the one or more registered users. The plurality of subsystems includes a professional development score generation subsystem. The professional development score generation subsystem is configured to generate professional development score for each of the user profile based on one or more factors upon completion of the one or more tasks.

The plurality of subsystems also includes a report generation subsystem. The report generation system is configurate to generate a report on each of the one or more tasks completed by the one or more registered users relating to the continuing industrial education content. The report generation system is also configurate to includes a publish the generated report to the one or more registered users and other industrial authorities via a communications network.

In accordance with one embodiment of the disclosure, a method for accreditation of industrial professionals is disclosed. The method includes creating a user profile for one or more registered users using one or more user credentials. The method also includes providing access to continuing industrial education content to the one or more registered users based on the created user profiles. The method also includes managing one or more tasks associated with the user profiles relating to the continuing industrial education content upon providing the access.

The method also includes generating a credit score for each of the user profiles based on the one or more tasks completed by the one or more registered users. The method also includes generating professional development score for each of the user profile based on one or more factors upon completion of the one or more tasks. The method also includes generating a report on each of the one or more tasks completed by the one or more registered users relating to the continuing industrial education content. The method also includes publishing the generated report to the one or more registered users and other industrial authorities via a communication network.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram illustrating an exemplary computing system for accreditation of industrial professionals in accordance with an embodiment of the present disclosure;

FIG. 2 is a block diagram illustrating an exemplary computing environment comprising a user device and a server device communicating through a communication network, in accordance with an embodiment of the present disclosure;

FIG. 3 is a block diagram illustrating another exemplary computing system for accreditation of industrial professionals in accordance with an embodiment of the present disclosure;

FIG. 4 illustrates a block diagram of components of the computing system and the interaction among themselves, in accordance with an embodiment of the present invention;

FIG. 5 is a flowchart illustrating an exemplary process of calculating the professional development score in accordance with an embodiment of the present disclosure;

FIG. 6 is a block diagram illustrating components in the computing system, such as those shown in FIG. 1, in accordance with an embodiment of the present disclosure; and

FIG. 7 is a process flowchart illustrating an exemplary method for accreditation of industrial professionals in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

A computer system (standalone, client or server computer system) configured by an application may constitute a “subsystem” that is configured and operated to perform certain operations. In one embodiment, the “subsystem” may be implemented mechanically or electronically, so a subsystem may comprise dedicated circuitry or logic that is permanently configured (within a special-purpose processor) to perform certain operations. In another embodiment, a “subsystem” may also comprise programmable logic or circuitry (as encompassed within a general-purpose processor or other programmable processor) that is temporarily configured by software to perform certain operations.

Accordingly, the term “subsystem” should be understood to encompass a tangible entity, be that an entity that is physically constructed permanently configured (hardwired) or temporarily configured (programmed) to operate in a certain manner and/or to perform certain operations described herein.

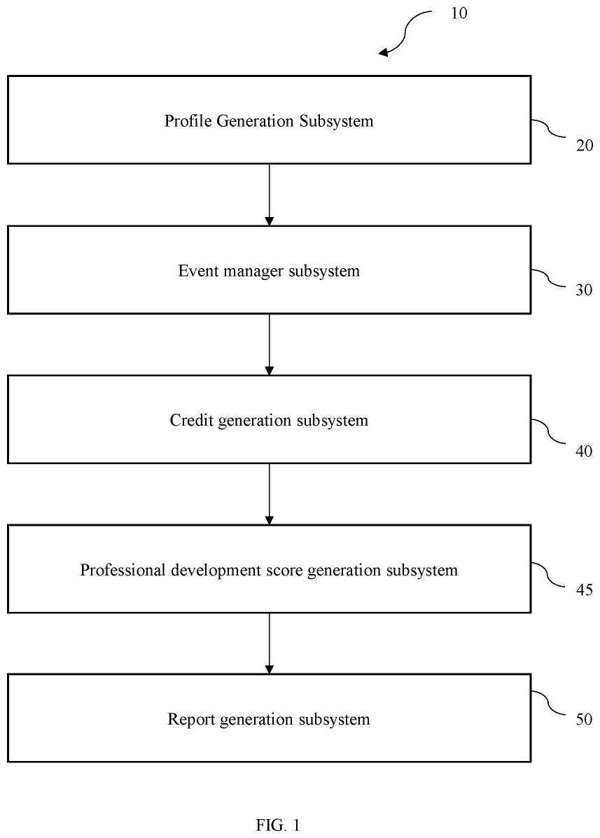

FIG. 1 is a block diagram illustrating an exemplary computing system 10 for accreditation of industrial professionals in accordance with an embodiment of the present disclosure. The system 10 provides a common platform to the industrial professionals and industrial organizations, such as hospitals, institutions, legal institutes, engineering institutes and the like to share and access information related to continuing industrial education, related activities occurring, and accreditation associated with attending continuing industrial education activities. Further, the system 10 allows the industrial professionals to apply with the continuing industrial education activities in order to obtain the accreditation and hence maintain their industrial licenses for practice.

The system 10 includes a hardware processor. The system 10 also includes a memory coupled to the hardware processor. The memory comprises a set of program instructions in the form of a plurality of subsystems. The plurality of subsystem is configured to be executed by the hardware processor.

The plurality of subsystems includes a profile generation subsystem 20. The profile generation subsystem 20 is configured to create a user profile for each of one or more registered users using one or more user credentials. In one embodiment, the one or more registered users belong to any industry. In such embodiment, the one or more registered users may be of two types, an industrial professional, such as a doctor, a nurse, a lawyer, an engineer and the like and an industrial organization such as a hospital, an institute, a nursing school, an engineering college, a law firm and the like. In such embodiment, two separate tabs are provided for logging in as a professional or as an organization.

The one or more user credentials of the one or more registered users' may include and is not restricted to personal details, professional career details, photographs, industrial experience with specialty, credits scores earned and are in need to renew licenses in case license is expired, continuing industrial education activities attended and/or performed by the industrial professional, their active industrial license number; status of their industrial practice such as active or expired license, and profile of the industrial organizations including contact details, continuing industrial education activities developed and delivered by the organization, credits earned by the organization for delivering the continuing industrial education activities, ranking of the organization and the like.

The system 10 provides various features such as view, access or update through such generated user profiles. The profile owner may automatically gain knowledge and awareness about currently occurring continuing industrial education events or activities and apply for attending them, which eventually helps them in obtaining the credit scores.

In one specific embodiment, each of the one or more registered users are assigned an identification label. This identification label is assigned based on information related to each of the one or more registered users, such as, but not limited to, name, contact details or industrial license number which uniquely identifies the one or more registered users. These are the one or more user credentials as provided during profile generation. The identification label may include and not limited to, a bar code, a quick response (QR) code, a numeric code, an alpha-numeric code, a graphical code and the like, which when scanned by an optical device is able to fetch the information related to each of the one or more registered users from a server device and identify each of the one or more registered users. Hence, it becomes digitally easier to identify who has attended or contributed or participated in a particular continuing industrial education activity, by scanning one of the one or more registered user's identification label at the time of registration for the continuing industrial education.

The plurality of subsystems includes an event manager subsystem 30. The event manager subsystem 30 is configured to provide access to continuing industrial education content to the one or more registered users based on the created user profiles. The system 10 continuously fetches matching information related to the continuing industrial education activities, based on each of the one or more registered user's preferences, and transmits such continuing industrial education activities to the corresponding one or more registered users.

In one specific embodiment, an information crawler parses an internet to scan, identify, and collect information related to the continuing industrial education content and associated activities occurring in a specified location. Such information needs to be presented on the system 10 for sharing among specific industrial community. Furthermore, the information crawler continuously gathers information related to the continuing industrial education content and associated activities from the internet. This information is stored in the database 180. Hence, the application 108 also collects the information about the continuing industrial education content and the related activities held by the organizations who are not the users of the application 108, avoiding the chances of missing out any important knowledge source or continuing industrial education activity occurrence.

The continuing industrial education content or related information may include and not limited to, general knowledge about the continuing industrial education, spreading awareness about the continuing industrial education program; activities such as live events, written publications, online programs, audio, video, or other electronic media and the like, industrial organization holding the activities for continuing industrial education with the information related to activities, industrial faculty and organizations developing, reviewing, and delivering contents about the continuing industrial education, and industrial bodies regulating continuing industrial education, and any other necessary information related to the continuing industrial education.

The event manager subsystem 30 is also configured to manage one or more tasks associated with the user profiles relating to the continuing industrial education content upon providing the access. The system 10 enables proper control over accessing such stated continuing industrial education content.

In such embodiment, the event manager subsystem 30 manages the one or more tasks for each of the one or more registered users respective to their user profiles. For example, the one or more tasks are related to their continuing industrial education and the associated activities, such as listing the conferences each of the one or more registered users is interested in, the time, date and place for each conference, and developing, delivering and organizing events by an associated organization, pushing them notifications or reminders for that event, providing a list of conferences, lectures happening around the industrial professional, or a list of publications to be launched on a particular date, or information such as time, date place of other such activities and informing the one or more registered users via notifications.

Additionally, the system 10 may also help the industrial professionals to directly apply online for the events they are interested to participate and/or perform an activity in the event, such as being a speaker. Directly applying may redirect each of the one or more registered users to a website of the organizations holding the event (whether live or online) or access a particular paper or journal for contribution. Further, the system 10 may provide an online form for the one or more registered users to fill in order to participate in such continuing industrial education activities. The continuing industrial education activities may also include delivering a lecture in an industrial institution, or writing and/or contributing to a journal, participating as a faculty or attending lectures in the industrial institution and the like. Such stated process includes enhancing knowledge of the industrial professional and also helps in maintaining their knowledge, skills, competence and performance. The event manager subsystem 30 is further configured to prompting the one or more registered users to renew an industrial license with the industrial organization based on a time of expiration of the industrial licenses.

At the time of participation in the registered continuing industrial education activity, the user may present his user device 52 in order to digitally scan his/her identification label 110 for marking his/her presence at the activity. Doing this, the presence of the user may be automatically identified during the start, the duration and the end of the activity. Resultantly, the application 108 may help in determining the presence of the user in the activity and give assurance to the organizing medical committee/organization of the user's presence throughout the activity after successful completion of the participation in the activity, the application 108 may also automatically awards the credit scores associated with that particular activity and adds them to the present credit score of the user. Hence, the system 10 automatically accredits the industrial professionals for their participation in the continuing industrial education activities and updates the renewal date of their medical licenses.

The plurality of subsystems also includes a credit generation subsystem 40. The credit generation subsystem 40 is configured to generate a credit score for each of the user profiles based on the one or more tasks completed by the one or more registered users. In one embodiment, each of the one or more registered users is given the credit score based on a number of meetings attended, or conferences participated in, or courses accessed. The credit score is awarded to the participating industrial professionals based on a verification process, either instantly or after sometime.

Exemplary verification criteria for providing the credit score awarded by the organizing industrial bodies or organization may depend upon a subject matter, status of a speaker and quality of papers to be presented in the conference or workshop or seminar or panel discussion. For example, the credit score may be calculated as 1 hour theory=1 credit hour and 1-hour hands on practice=2 credit hours and so on. Other exemplary verification criteria for providing the credit score may be such as, for each state or national or international, maximum credit hours is awarded as per the schedule of the conference for participation: (a) Half Day—4 credit hours; (b) One day conference 8 credit hours; (c) Two days conference 16 credit hours; (d) Three days conference 24 credit hours and so on.

Further, examples of verification criteria for providing the credit scores may be any paper published in indexed state or national or international journal entitles an author or co-author for the credit hours. Alternatively, any chapter published in a textbook or update book published by professional bodies entitles the author or co-authors for the credit hours. In another alternate embodiment, speakers or chairman or co-chairman or moderator at any program is given two credit hours per talk in addition to the credit hours allotted for that particular academic activity. An organizer of the program except in-house program is awarded five credit hours and an organizer of the in house program is awarded two credit hours. In such embodiment, the paper presentation (oral or poster) in international conferences is awarded six credit hours and in state or national conferences is awarded four credit hours. Alternatively, if the conference is a group work, only first and second investigators shall share the credit hours of six hours; and four hours, if the conference is of state or national conference. Hence, there may be a number of ways for deciding the verification criteria for accreditation by the industrial organization developing and delivering the continuing industrial education.

When all the users, who are the industrial professionals, have successfully completed their participation or contribution in the continuing industrial education activity, the system 10 may also automatically have a list of the industrial professionals who have attended the particular activity and send that list to the particular industrial organization who has held that activity. This can be done by scanning the identification labels of the industrial professionals. Hence, the industrial organization will automatically have a list of attending professionals with their details and industrial license numbers, hence avoiding the hectic use of paperwork. Furthermore, the credit scores are awarded to the participating professionals based on the verification, either instantly or after some time.

The plurality of subsystems also includes a professional development score generation subsystem 45. The professional development score generation subsystem 45 is configured to generate professional development score for each of the user profile based on one or more factors upon completion of the one or more tasks. In one embodiment, the one or more factors comprises continuing industrial education history, number of years of schooling and college education, total levels of continuing professional development courses, employment history, types of certifications undertaken and number of associated conferences attended, number of associated white papers published and the like.

The professional development score plays a key role in a talent manager's decision to offer a job and may be used by many organizations to institutions. One metric primarily used in calculating a professional development score is knowledge utilization or the percentage of education currently being used in an individual's respective professional experience. In one specific embodiment, a professional development score of 85 or above is generally considered good and may result in a professional receiving a better job profile. Scores greater than 85 are considered excellent. Scores between 70 to 84 are considered to be good. Scores between 69 to 55 are considered to be fair. Scores between 54 to 30 are considered to be fair. Scores between 25 to 10 are considered to be very poor. An individual's professional development score provides statistical analysis of skill worthiness and directly affects professional prospects and pay scale.

Furthermore, the professional development score changes and may rise or fall based on new information. In such embodiment, the professional development score changes due to updating of knowledge on time, by organising knowledge transfer initiatives, by attending more courses and by working with specialized experts to improve professional development score.

The plurality of subsystems also includes a report generation subsystem 50. The report generation system 50 is configured to generate a report on each of the one or more tasks completed by the one or more registered users relating to the continuing industrial education content.

The report generation system 50 is also configured to publish the generated report to the one or more registered users and other industrial authorities via a communication network. In such embodiment, each of the one or more registered user may access on the generated report via the user profiles.

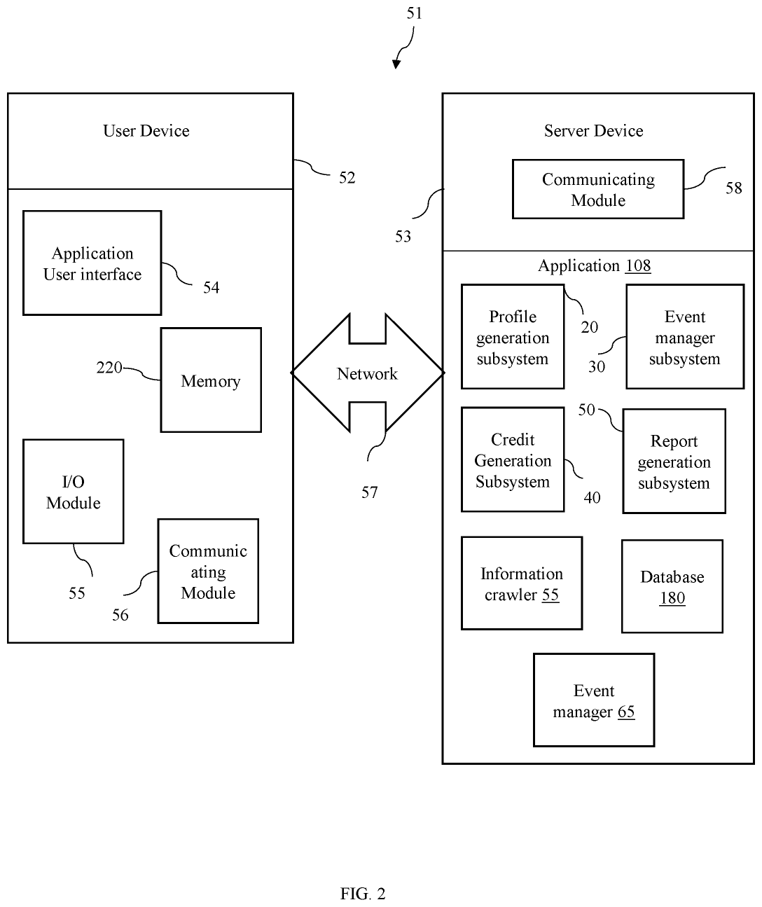

FIG. 2 is a block diagram illustrating an exemplary computing environment 51 comprising a user device 52 and a server device 53 communicating through a communication network 57, in accordance with an embodiment of the present disclosure.

In such embodiment, an industrial professional views and accesses continuing industrial education related information through the user device 52. The user device 52 may include an electronic communication device such as a computer, a laptop, a smart phone, a tablet and the like. In one such embodiment, the user device 52 may run software programs such as an embedded web browser or custom client application, capable of communicating with the server device 53 or input/output module 55. Further, the communication network 57 may include, but is not restricted to, Internet, PSTN, Local Area Network (LAN), Wide Area Network (WAN), and Metropolitan Area Network (MAN).

The computing environment 51 provides an application 108 (also referred to as ‘app’ throughout the detailed description). The application 108 may be a mobile application or a web-based application or both that is run on the server device 53 and also accessible at the user device 52. The application 108 is operated by a user utilizing the user device 52. According to the present disclosure, the application 108 provides an interactive platform which is accessible to the industrial community, including the industrial professionals, such as and not limited to doctors, nurses, pharmacists, engineer, lawyer, or any other industry; and also the medical organizations such as and not limited to hospitals, institutions, nursing schools, other industrial organizations for sharing and accessing information related to the continuing industrial education which is necessary for the industrial professionals to enhance their industrial knowledge throughout their tenure. The continuous industrial education related information may include and not limited to general knowledge about the continuous industrial education spreading awareness about the continuous industrial education program; activities such as live events, written publications, online programs, audio, video, or other electronic media and the like; industrial organization holding the activities for continuous industrial education with the information related to activities; medical faculty and organizations developing, reviewing, and delivering contents about the continuous industrial education; and industrial bodies regulating the continuous industrial education; and any other necessary information related to the continuous industrial education.

The user device 52 includes an application user interface 54, a memory 220, an input/output module 55 (I/O module), and a communicating module 56. The application 108 has the user interface 54 displayed at the user device 52 through which the user may browse for operating the application 108 using the I/O module 55. The I/O module 55 may include and is not restricted to keyboard, touch pad, mouse, camera, speaker, microphone, display screen and the like. The user device 52 has the memory 220 for running and processing the application 108, if the application 108 is a mobile application, and also stores other entities, such as software programs to run the application 108. Further, the user device 52 communicates with the server device 53 using the communicating module 56 via the network 57.

The server device 53 includes a communicating module 58. The application 108 executes on the server device 53. The application 108 consists of a profile generation subsystem 20, a credit generating subsystem 40, an event manager subsystem 30, a report generation subsystem 50, an information crawler 55, an event manager 65; and a number of databases 180. The user creates a profile on the application 108 using the profile generation subsystem 20. The credit generating subsystem 40 calculates credit scores to be awarded to the user including the industrial professional and/or the organization based on their participation in continuous industrial education activities or organizing such activities. The information crawler 55 parses internet for crawling, scanning, identifying and collecting information related to the continuous industrial education and associated activities occurring in the states, the country or abroad. Such information needs to be presented on the application 108 for sharing among the industrial community.

The event manager 65 manages the one or more tasks for the users respective to their profiles related to their continuous industrial education and the associated activities. The communicating module 58 allows the server device 53 to communicate with the user device 52.

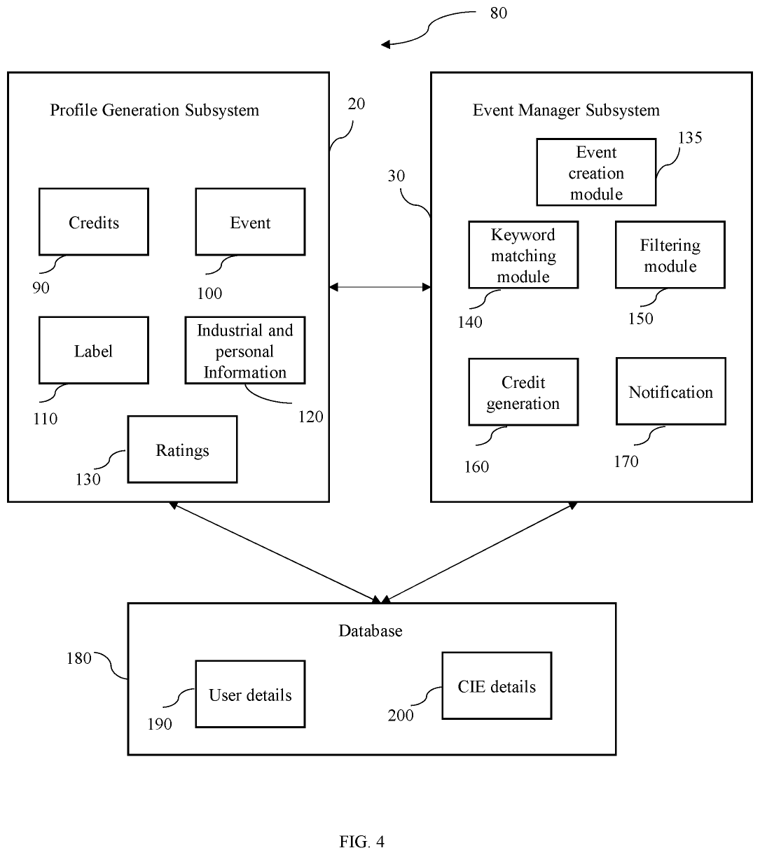

The application 108 comprises a number of databases 180 at the server device 53 that stores all the information regarding the users (professionals and organizations) who have created profiles at the application 108; and also about all the information provided by the regulatory and/or industrial organizations about the continuous industrial education and associated activities; and the information parsed by the information crawler 55 from the internet; and any other necessary information for the knowledge of the continuous industrial education. The database 130 hence stores and maintains information related to the users including the professionals and the organizations and all the necessary information about the continuous industrial education which can inform the users about any activities occurring and educate the users about the necessity of the continuous industrial education. A detailed description about the databases 180 is provided in FIG. 4.

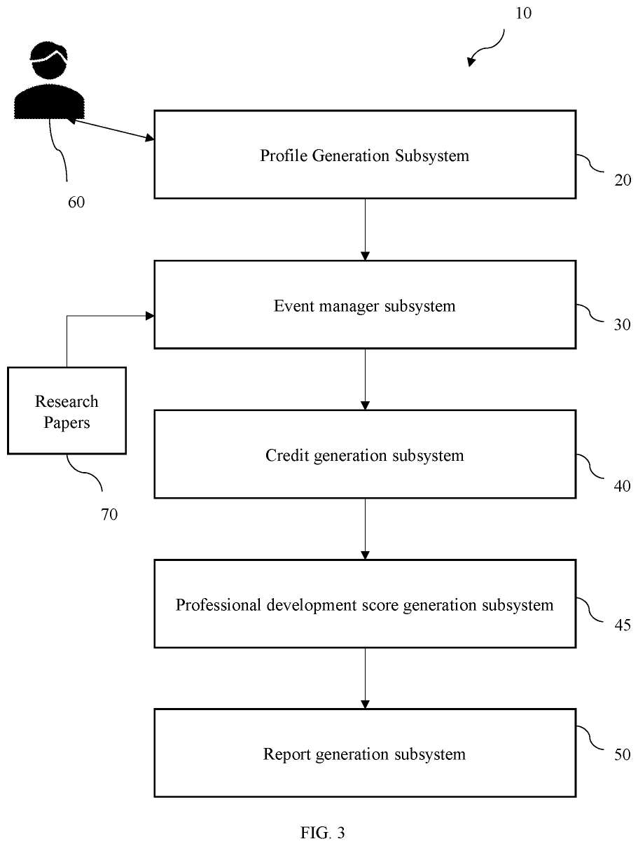

FIG. 3 is a block diagram illustrating another exemplary computing system 10 for accreditation of industrial professionals in accordance with an embodiment of the present disclosure. In the given exemplary embodiment, a medical professional X 60 working in a hospital Y registers in the system 10 to access and learn about new research papers 70 published in a particular health domain. The medical professional X 60 first registers in the system 10 and creates a profile through a profile generation subsystem 20. The medical professional X 60 provides valid credentials during the registration and creation of the user profile.

The medical professional X 60 is provided access to the particular research papers 70 via an event manager subsystem 30. For example, the event manager subsystem 30 may provide access to the research papers 70 on the medical journal based on a particular subject matter. The system 10 via the event manager subsystem 30 further manages one or more tasks of the medical professional X 60 via the generated user profile of the medical professional X 60.

The system 10 via a credit generation subsystem 40 may also generate a credit score for the one or more tasks as executed by the medical professional X 60 while accessing the particular research papers 70. A professional development score is generated by a professional development score generation subsystem 45 for each of the user profile based on one or more factors upon completion of one or more tasks. The credit scores may be used for certification purpose of the medical professional X 60. The system 10 further generates and publishes a report of the one or more tasks via a report generated subsystem 50.

Similarly, the system 10 may behave similarly for any engineering professional or a legal professional, who wishes to get accreditation for professional work. The engineering professional may undergo the industrial education courses as provided by the system 10 and earn requisite credit score for gaining accreditation or certificate for any course or degree.

The profile generation subsystem 20, the event manager subsystem 30, the credit generation subsystem 40 and the report generation subsystem 50 in FIG. 3 is substantially equivalent to the profile generation subsystem 20, the event manager subsystem 30, the credit generation subsystem 40 and the report generation subsystem 50 of FIG. 1.

FIG. 4 illustrates a block diagram of components of the computing system 80 and the interaction among themselves, in accordance with an embodiment of the present invention. The profile generation subsystem 20 is configured to access personal and industrial information 120 from the each of the one or more registered users. During the process of operation of the system 80, the profile generation subsystem 20 helps to manipulate or generate events 100, credit 90, label 110 and ratings 130.

In an embodiment, a user profile briefs an overview of his/her career and personal details. The profile generation subsystem 20 creates a user profile page dedicated to the user that shows user's personal and professional details along with the credit scores awarded to the user. The user profile includes and not limited to the personal and professional details of the user 120; credit scores earned 90; a list of CME activities/events participated 100; professional status of the user; identification label of the user 110, and the ratings 130. While creating the profile at the application 108, the application 108 at the first may ask the user whether they are an industrial professional or an organization. This way the application 108 stores the information separately for the industrial professionals and the industrial organizations at the server device 53. Thereafter, the user may fill into the details as asked and aforementioned. An industrial professional has to enter his/her industrial license number, which can indicate his/her industrial status for industrial practice. As the profile is created, the application 108 automatically creates an identification label 110 dedicated to the particular user. The identification label 110 is any mark or representation that contains information related to the user, such as and not limited to name, contact details or industrial license number which uniquely identifies the user. The label 110 may include and not limited to a bar code, a QR code, a numeric code, an alpha-numeric code, a graphical code and the like, which when scanned by an optical device is able to fetch the details from the server device 53 and identify the user. Therefore, any user who creates the profiles at the application 108 can be uniquely identified. Hence, it is digitally easier to identify who has attended or contributed or participated in a particular continuous industrial education activity, by scanning their identification label 110 at the time of registration for the continuous industrial education activity.

Further in an embodiment, the profile generation module 20 may also provide ratings 130, in case of industrial organizations depending on their reputations and contributions to the industrial community and also depending on the number and frequency of the continuous industrial education activities developed and delivered by them.

The application 108 also includes an event manager subsystem 30 which communicates with the profile generation module 20 and manages and communicates necessary tasks or information related to the continuous industrial education content and associated activities to the users. The application 108 continuously stores information related to the users and the continuous industrial education activities in its databases 180. Since, it is obvious that a particular industrial professional with his/her specialty will only be interested to attend or contribute the activity of the continuous industrial education which is either dedicated to his/her specialty or somehow related to the specialty. Therefore, if all the information about every continuous industrial education activity is provided to every user in the application 108 page, then it may or may not be considered as non-useful or spam info. Hence, the event manager subsystem 30 uses an event creation module 135 to create and store a list of related activities helpful for a user to attend.

The event manger subsystem 30 is configured to manage each of the events of the system 80. Keyword matching module 140 and filtering module 150 are used to fetch a list of matching continuing industrial education activities/events and provide them to each of the one or more registered users. The keyword matching module 140 and the filtering module 150 maps the user's profile which briefs about his/her industrial professional career, and his medical interests provided in the profile, which may or may not be related to his professional domain, with a list of continuous industrial education activities (and/or information) currently happening or about to happen in future, which is available for participation. Further, in an embodiment, the keyword matching module 140 may also present the users with a list of continuous industrial education activities based on their profiles and location and other interests listed in the profile. Hence, the application 108 provides the users with the list of continuous industrial education activities occurring around them. The event manager subsystem 30 sends the list of continuous industrial education activities and information in users' inboxes or messages (not shown in the Figures), from where the users can access the link and apply for registration of the activities.

A notifications subsystem 170 alert the one or more registered users about event details. The notification subsystem 170 may also automatically create a calendar event based on the registration of the user for a particular activity and may also remind the user as the activity time and date approaches. The notifications may be in the form of a message, a call, an email, a calendar reminder or the like.

Furthermore, all the user details 190 and the continuing industrial education details 200 are stored in a database 180. The database 180 may be remotely stored or locally stored. The database 180 stores information related to each of the one or more registered users including the industrial professionals and the organizations and all the necessary information about the continuing industrial education which may inform the each of the one or more registered users about any activities occurring and educate each of the one or more registered users about the necessity of the continuing industrial education.

For the whole process to operate, the system 80 is first integrated into various training platforms and then a user logs in and performs training to get accreditation. Initially, an industry specific cabinet first approves or appoints a sole continuous professional development (CPD) reckoner. The appointed continuous professional development (CPD) reckoner directs industry specific councils to integrate various system portal shortcuts in their customized websites for access.

The appointed continuous professional development (CPD) reckoner also creates an advisory board to help the system 80 with achieving set targets. In one embodiment, a group of users acting as an advisory board is created, which further comprises a plurality of subject matter experts. The advisory board provides license renewal support with industry specific cabinet. In one specific embodiment, the plurality of subject matter experts comprises five to seven subject matter experts.

In one embodiment, a plurality of knowledge or technical providers approvals and license renewal support is dynamically linked with industry specific councils. In one such embodiment, at least one member of the advisory board comprises a member from the respective industry specific councils. Moreover, one or more scope of work contracts is generated for each group of users acting as an advisory board member.

The system 10 manages one or more tasks by the plurality of subject matter experts for on boarding of all the plurality of knowledge or technical providers. In such embodiment, a number pre-defined continuous professional development (CPD) programs are registered. In another such embodiment, all knowledge or training providers are made acquainted with sponsorship opportunity and supports. Various sponsorship options as provided by all knowledge or training providers have three months lead time. Furthermore, one or more marketing and public relation activities for the one or more tasks is generated based on the continuous professional development (CPD) programs.

In such exemplary embodiment, the system 80 captures one or more user details for registration of a user and for creation of customized user profile dashboard. The one or more user details comprises at least one of electronic mail, mobile number, practise license, photos and the like. In such embodiment, a payment gateway is dynamically linked for payment from one or more registered user.

In one embodiment, the system 80 enables access of a customized user profile dashboard to one or more registered user after payment. The customized user profile dashboard ensures easy search for continuous professional development (CPD) programs and post continuous professional development (CPD) programs follow ups. In such embodiment, the customized user profile dashboard is navigated with options based on city, zip code, specialty, date, topic and industry affiliation and to choose at least on of continuous professional development (CPD) programs. Against each continuous professional development (CPD) programs credit points along with confirmation possibilities are provided. In such embodiment, the access of the chosen continuous professional development (CPD) programs enables accumulation of credit points.

The knowledge and training programs are listed and when confirmed by the one or more registered users reading materials are provided through email ID. All notifications and updates are sent via the electronic mail. In such embodiment, the industrial activity is attended by the each of the one or more registered users via access code entry. Once assessments are done, the credit scores are provided to each of the one or more registered users.

Additionally, the system 80 provides easy process for license renewal. In one embodiment, each of the one or more registered users is enabled to access and train with the chosen continuous professional development (CPD) programs for a pre-defined period of time to achieve a set value of the credit points. For example, the pre-defined period of time period of time may be five years and the set value of the credit points may be 30 credit points.

In such embodiment, upon achieving the set value of the credit points, each of the one or more registered users is allowed to submit renewal application online form and documents. The documents comprise scanned undertaking certificate, two pass-port size photos, and self-attested permanent registration certificate and the like.

In another embodiment, the training with the chosen continuous professional development (CPD) programs for each of the one or more registered users for the pre-defined period of time is reauthenticated before submission of the renewal application online form and the documents over to the industry specific councils. The industry specific councils approve license after payment of renewal fees and reauthentication. In such embodiment, the system 80 alerts the one or more registered users for download of colour copy of the renewed industrial license upon approval from the industry specific council. Alerts may be provided through electronic mail, mobile messages and the like. The cycle of accumulation of points is repeated for another pre-defined period of time.

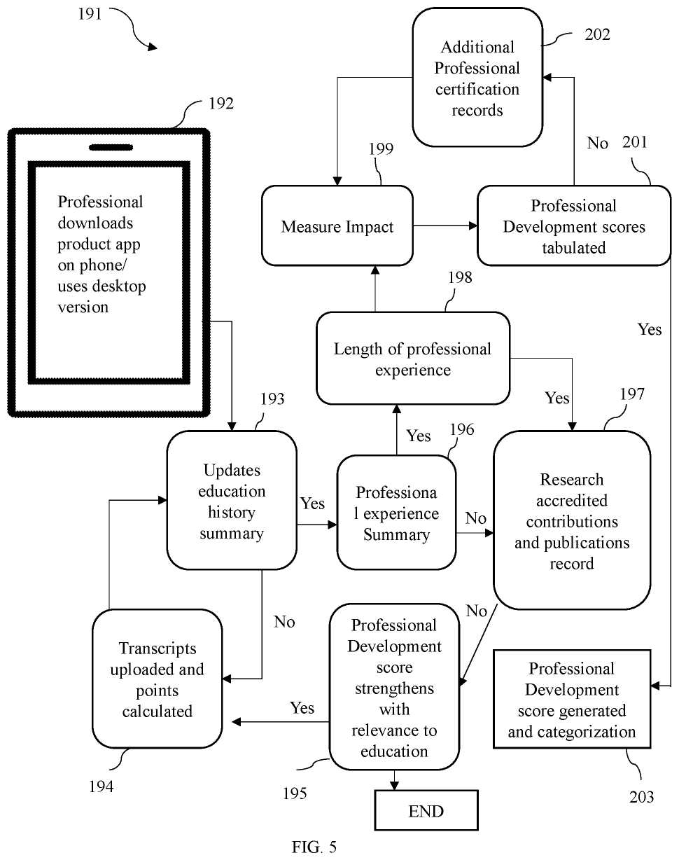

FIG. 5 is a flowchart illustrating an exemplary process 191 of calculating the professional development score in accordance with an embodiment of the present disclosure. At the step 192, application of the system 10 is downloaded in a phone or any handheld device by each of the one or more registered user. A desktop version may also be downloaded by Each of the one or more registered user.

At the step 193, education history summary of each of the one or more registered user is updated. In such embodiment, at the step 194, transcripts are updated, and points are calculated if the education history summary of each of the one or more registered user is not updated.

At the step 196, professional experience summary is generated for each of the one or more registered user. Further, at the step 198, length of professional experience is calculated for each of the one or more registered user. In such embodiment, at the step 199, an impact is measured from the calculated length of professional experience and additional professional certification records. At the step 201, the professional development score is tabulated based on the measured impact. At the step 203, the professional development score is generated and categorized from the tabulated professional development score. Moreover, at the step 202, more additional professional certification records are accepted to re-measure the impact.

At the step 197, research accredited contributions and publications records are accepted from each of the one or more registered users to generate professional experience summary. In such embodiment, at the step 195, the contribution relevant to education is only used to strengthen the professional development score.

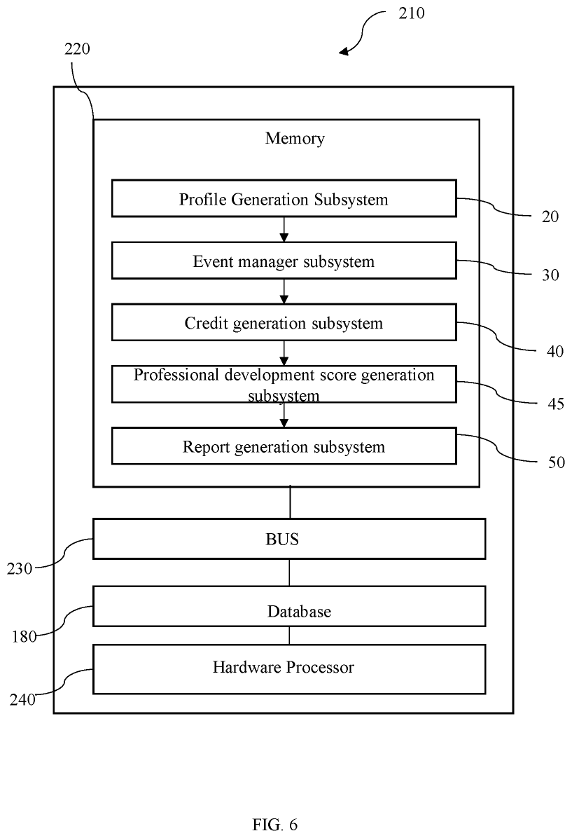

FIG. 6 is a block diagram illustrating components in the computing system 210, such as those shown in FIG. 1, in accordance with an embodiment of the present disclosure. The components comprise a hardware processor 240, a memory 220, a database 180 and a bus 230.

The hardware processor(s) 240, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 220 includes a plurality of subsystems stored in the form of executable program which instructs the hardware processor 240 via bus 230 to perform the method steps as illustrated in FIG. 1. The memory 220 has following subsystems: the profile generation subsystem 20, the event manager subsystem 30, the credit generation subsystem 40, professional development score generation subsystem 45 and the report generation subsystem 50. The database 180 is configured to store each of the one or more registered users' information and continuing industrial education content.

The profile generation subsystem 20 is configured to create user profiles for one or more registered users using one or more user credentials. The event manager subsystem 30 is configured is provide access to continuing industrial education content to the one or more registered users based on the created user profiles. The event manager subsystem 30 is also configured is manage one or more tasks associated with the user profiles relating to the continuing industrial education content upon providing the access.

The credit generation subsystem 40 is configured to generate a credit score for the user profiles based on the one or more tasks completed by the one or more registered users. The professional development score generation subsystem 45 is configured to generate professional development score for each of the user profile based on one or more factors upon completion of the one or more tasks. The report generation system 50 is configured to generate a report on each of the one or more tasks completed by the one or more registered users relating to the continuing industrial education content. The report generation system 50 is also configured to publish the generated report to each of the one or more registered users and other industrial authorities via the communication network 57.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor(s) 240.

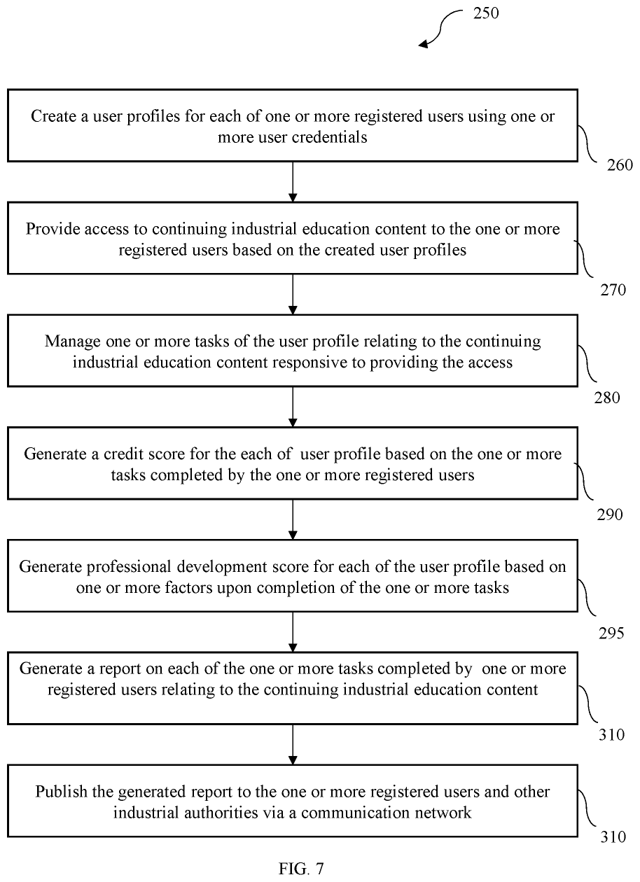

FIG. 7 is a process flowchart illustrating an exemplary method 250 for accreditation of industrial professionals in accordance with an embodiment of the present disclosure. At step 260, a user profile is created for each of one or more registered users using one or more user credentials. In one aspect of the present embodiment, the user profile is created for each of the one or more registered users by a profile generation subsystem 20.

At step 270, access is provided to continuing industrial education content to the one or more registered users based on the created user profiles. In one aspect of the present embodiment, access is provided to the continuing industrial education content by an event manager subsystem 30.

At step 280, one or more tasks associated with the user profile relating to the continuing industrial education content is managed upon providing the access. In one aspect of the present embodiment, the one or more tasks associated with the user profile relating to the continuing industrial education content is managed by an event manager subsystem 30.

The method 250 also includes providing the access to the continuing industrial an education content to the one or more registered users comprises using a keyword searching technique and a filtering technique. The method also includes prompting the one or more registered users to renew an industrial license with an industrial organization based on time of expiration of the industrial licenses.

At step 290, a credit score for each of the user profile are generated based on the one or more tasks completed by the one or more registered users. In one aspect of the present embodiment, the credit score for each of the user profile are generated based on the one or more tasks completed by the one or more registered users by a credit generation subsystem 40.

At step 295, a professional development score is generated for each of the user profile based on one or more factors upon completion of the one or more tasks. In one aspect of the present embodiment, the professional development score is generated for each of the user profile based on the one or more factors upon completion of the one or more tasks by a professional development score generation subsystem 45. In another aspect of the present embodiment, the one or more factors comprises continuing industrial education history, number of years of schooling and college education, total levels of continuing professional development courses, and employment history.

At step 300, a report is generated on each of the one or more tasks completed by the one or more registered users relating to the continuing industrial education content. In one aspect of the present embodiment, the report is generated on the one or more tasks completed by the one or more registered users relating to the continuing industrial education content by a report generation subsystem 50.

At step 310, the generated report is published to the one or more registered users and other industrial authorities via a communications network. In one aspect of the present embodiment, the generated report is published to the one or more registered users and other industrial authorities by the report generation subsystem 50.

The method 250 includes scanning, identifying and collecting the continuing industrial education content using an information crawler. In one embodiment, the one or more tasks of the user profiles comprises listing the conferences each of the one or more registered users is interested in, time, date, and place for that conference, and developing, delivering and organizing events by an industrial representative of any associated organization. Furthermore, the method includes storing both information related to each of the one or more registered users and information related to the continuing industrial education content.

The method 250 for managing one or more tasks associated with the user profile comprises approving and appointing a sole continuous professional development (CPD) reckoner. In such embodiment, the appointed continuous professional development (CPD) reckoner directs industry specific councils to integrate various system portal shortcuts in their customized websites for access. Further, the appointed continuous professional development (CPD) reckoner creates an advisory board to help with achieving set targets. Specific number of knowledge and training programs are planed with the help of subject matter experts, wherein all knowledge and training providers are made acquainted with sponsorship opportunity and supports.

The method 250 for creating a user profile for each of one or more registered users comprises registering for the user profile through an electronic mail ID and a mobile number. In one embodiment, customized dashboard is accessed after payment via a third-party application. In such embodiment, the customized dashboard is navigated with options based on location, date, topic and industry affiliation.

The method 250 for renewal of an industrial license comprises training for certain number of years to fulfil number of requirement years. In such embodiment, after such training necessary documents are submitted for the industrial license renewal to the industry specific council. For renewable of an industrial license, required renewal fees is paid through a payment gateway.

In such embodiment, the continuing industrial education content comprises knowledge regarding the continuing industrial education content, spreading awareness regarding a continuing industrial education program, activities of live events, written publications, online programs, audio, video, and other electronic media and activities comprising developing, reviewing, and delivering contents regarding continuing industrial education.

The present invention provides an interactive platform which is common between the industrial professionals and the industrial organizations and provides them a common provision to share, view and access all the available information related to the continuing industrial education awareness and activities for continuing industrial education. Further, the system thereof provides an automated provision for the industrial professionals to view and be updated with the current knowledge of their industrial fields and also about the continuing industrial education activities. Furthermore, the system provides an automated way of getting updates about the continuing industrial education activities may or may not be based on their preferences, registering for a desired continuing industrial education activity and obtains the credit scores related to that activity. The system provides a professional development score, which may help a professional on career path through their lifetime.

Additionally, the system also provides an automated platform for the industrial bodies to issue notices about the number of continuing industrial education activities they are organizing and call for participants either by announcing the notifications on the interface of the system, or by pushing notifications to the preferred professionals. Additionally, the system provides the organizing industrial bodies an automated and digitized way of accreditation which avoids the needs of hectic paperwork and organizing things on paper. Thereby, the system provides industrial education compliance platform along with centralized tracking and support system. The system can easily track fees and attendance of any registered user.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A system for accreditation of industrial professionals, the system comprising:
a hardware processor; and
a memory coupled to the hardware processor, wherein the memory comprises a set of program instructions in the form of a plurality of subsystems, configured to be executed by the hardware processor, wherein the plurality of subsystems comprises:
a profile generation subsystem configured to create a user profile for each of one or more registered users using one or more user credentials, wherein the one or more registered users belong to one or more industries;
an event manager subsystem configured to:
provide access to continuing industrial education content to the one or more registered users based on the created user profile; and
manage one or more tasks associated with the user profile relating to the continuing industrial education content responsive to providing the access;
a credit generation subsystem configured to generate a credit score for each of the user profile based on the one or more tasks completed by the one or more registered users;
a professional development score generation subsystem configured to generate professional development score for each of the user profile based on one or more factors upon completion of the one or more tasks, wherein the one or more factors comprises continuing industrial education history, number of years of schooling and college education, total levels of continuing professional development courses, and employment history; and
a report generation subsystem configured to:
generate a report on each of the one or more tasks completed by the one or more registered users relating to the continuing industrial education content; and
publish the generated report to the one or more registered users and other industrial authorities via a communications network.

2. The system of claim 1, wherein the system further comprises an information crawler configured to scan, identify, and collect information related to the continuing industrial education content and associated activities occurring in a specified location.

3. The system of claim 1, wherein the one or more tasks of the user profiles comprises listing conferences each of the one or more registered users is interested in, a time, a date and a place for each conference, and developing, delivering and organizing events by an industrial representative of an associated organization.

4. The system of claim 1, wherein the credit score is generated based on a number of meetings, conferences and courses accessed by each of the one or more registered users.

5. The system of claim 1, wherein the access to the continuing industrial education content to the one or more registered users is provided by using a keyword searching technique and a filtering technique.

6. The system of claim 1, further comprising a database configured to store information related to each of the one or more registered users and information related to the continuing industrial education content.

7. The system of claim 1, wherein the continuing industrial education content comprises continuing medical education.

8. The system of claim 1, wherein the profile generation subsystem is further configured to assign an identification label to each of the created user profile, and wherein the identification label comprises contact details of the one or more registered users and an industrial license number.

9. The system of claim 1, wherein the continuing industrial education content comprises knowledge regarding continuing industrial education content, spreading awareness regarding a continuing industrial education program, activities of live events, written publications, online programs, audio, video, and other electronic media and activities comprising developing, reviewing, and delivering content regarding continuing industrial education.

10. The system of claim 1, wherein the event manager subsystem is further configured for prompting the one or more registered users to renew an industrial license with an industrial organization based on a time of expiration of the industrial licenses.

11. A method for accreditation of industrial professionals, the method comprising:
creating, by a processor, a user profile for each of one or more registered users using one or more user credentials, wherein the one or more registered users are associated with one or more industries;
providing, by the processor, access to continuing industrial education content to the one or more registered users based on the created user profile;
managing, by the processor, one or more tasks associated with the user profile relating to the continuing industrial education content responsive to providing the access;
generating, by the processor, a credit score for each of the user profile based on the one or more tasks completed by the one or more registered users;
generating, by the processor, professional development score for each of the user profile based on one or more factors upon completion of the one or more tasks, wherein the one or more factors comprises continuing industrial education history, number of years of schooling and college education, total levels of continuing professional development courses, and employment history;
generating, by the processor, a report on each of the one or more tasks completed by the one or more registered users relating to the continuing industrial education content; and
publishing, by the processor, the generated report to the one or more registered users and other industrial authorities via a communications network.

12. The method of claim 11, further comprising scanning, identifying and collecting information related to the continuing industrial education content and associated activities occurring in a specified location using an information crawler.

13. The method of claim 11, wherein the one or more tasks of the user profiles comprises listing the conferences each of the one or more registered users is interested in, a time, a date, and a place for each conference, and developing, delivering and organizing events by an industrial representative of associated organization.

14. The method of claim 11, wherein the credit score is generated based on a number of meetings, conferences and courses accessed by each of the one or more registered users.

15. The method of claim 11, further comprising storing information related to each of the one or more registered users and information related to the continuing industrial education content in a database.

16. The method of claim 11, wherein the continuing industrial education content comprises continuing medical education.

17. The method of claim 11, further comprising assigning an identification label to each of the created user profile, wherein the identification label comprises contact details of the one or more registered users, and an industrial license number.

18. The method of claim 11, wherein the continuing industrial education content comprises knowledge regarding the continuing industrial education content, spreading awareness regarding a continuing industrial education program, activities of live events, written publications, online programs, audio, video, and other electronic media and activities comprising developing, reviewing, and delivering contents regarding continuing industrial education.

19. The method of claim 11, wherein providing the access to the continuing industrial an education content to the one or more registered users comprises providing the access using a keyword searching technique and a filtering technique.

20. The method of claim 11, further comprising prompting the one or more registered users to renew an industrial license with an industrial organization based on a time of expiration of the industrial licenses.

21. The method of claim 11, wherein for managing the one or more tasks associated with the user profile comprises:
creating a group of users acting as an advisory board comprising a plurality of subject matter experts, wherein at least one member of the advisory board comprises a member from respective industry specific councils;
dynamically linking a plurality of knowledge and technical providers approvals and license renewal support with industry specific councils, generating one or more scope of work contracts for each group of users acting as an advisory board member;
managing one or more tasks by the plurality of subject matter experts for on boarding of the plurality of knowledge and technical providers;
registering for a number of pre-defined continuous professional development (CPD) programs; and
generating one or more marketing and public relation activities for the one or more tasks based on the continuous professional development (CPD) programs.

22. The method of claim 11, wherein creating the user profile for each of the one or more registered users comprises:
capturing one or more user details for registration of a user and for creation of customized user profile dashboard, wherein the one or more user details comprises at least one of electronic mail, mobile number, practise license and photos;
dynamically linking a payment gateway for payment from the one or more registered user;
enabling access of a customized user profile dashboard to one or more registered user after payment, wherein the customized user profile dashboard ensures easy search for continuous professional development (CPD) programs and post continuous professional development (CPD) programs follow ups;
navigating the customized user profile dashboard with options based on city, zip code, specialty, date, topic and industry affiliation to select at least on of continuous professional development (CPD) programs; and
enabling access of the selected continuous professional development (CPD) programs for accumulation of credit points.

23. The method of claim 23, further comprises renewing an industrial license for the one or more registered users by:
enabling access to each of the one or more registered users and train the one or more registered users with the selected continuous professional development (CPD) programs for a pre-defined period of time to achieve a set value of the credit points;
allowing each of the one or more registered users to submit renewal application online form and documents upon achieving the set value of the credit points, wherein the documents comprise at least one of scanned undertaking certificate, two pass-port size photos, and self-attested permanent registration certificate;
reauthenticating the training with the chosen continuous professional development (CPD) programs for the pre-defined period of time before submission of the renewal application online form and the documents over to the industry specific councils, wherein the industry specific councils approve license after payment of renewal fees and reauthentication; and
alerting the one or more registered users for downloading a colour copy of renewed industrial license upon approval from the industry specific council.

24. A non-transitory computer-readable storage medium having instructions stored therein that, when executed by a hardware processor, cause the processor to perform method steps comprising:
creating, by a processor, a user profile for each of one or more registered users using one or more user credentials, wherein the one or more registered users are associated with one or more industries;
providing, by the processor, access to continuing industrial education content to the one or more registered users based on the created user profile;
managing, by the processor, one or more tasks of the user profile relating to the continuing industrial education content responsive to providing the access;
generating, by the processor, a credit score for each of the user profiles based on the one or more tasks completed by the one or more registered users;
generating, by the processor, professional development score for each of the user profile based on one or more factors upon completion of the one or more tasks, wherein the one or more factors comprises continuing industrial education history, number of years of schooling and college education, total levels of continuing professional development courses, and employment history;
generating, by the processor, a report on the one or more tasks completed by the one or more registered users relating to the continuing industrial education content; and
publishing, by the processor, the generated report to the one or more registered users and other industrial authorities via a communication network.
